(19) United States
(12) Patent Application Publication
(10) Pub. No.: US 2020/0246489 A1
(43) Pub. Date: Aug. 6, 2020

(54) DETECTING ARCHITECTURAL REMODELLING IN CELLS, EXTRACELLULAR MATRIX, AND THE TISSUE MICROENVIRONMENT

(71) Applicant: GLYMPSE BIO, INC., Cambridge, MA (US)

(72) Inventors: Sangeeta Bhatia, Lexington, MA (US); Gabriel Kwong, Atlanta, GA (US); Eric Huang, Lexington, MA (US); Sirshendu Roopom Banerjee, Lexington, MA (US); Andrew David Warren, Cambridge, MA (US); Sophie Cazanave, Cambridge, MA (US)

(21) Appl. No.: 16/777,025

(22) Filed: Jan. 30, 2020

Related U.S. Application Data
(60) Provisional application No. 62/800,100, filed on Feb. 1, 2019.

(51) Int. Cl. A61K 49/00; C12Q 1/37; A61B 6/00

(52) U.S. Cl. CPC A61K 49/0002; A61B 6/481; C12Q 1/37

(57) ABSTRACT

Methods of the present invention predict a physiological state of a subject. A composition comprising activity sensors is introduced into a body of a subject. The activity sensors comprise a plurality of reporters susceptible to cleavage when processed by the body during extracellular matrix remodeling. Cleavage may be indicative of enzymatic activity in the extracellular matrix. Signals detected by the activity sensors may be predictive of a physiological state. A sample is collected from the subject, and liberated reporters are detected in the sample. An onset of the physiological state of the subject, which may be a disease characterized by inflammation or atrophy, may be diagnosed based on the liberated reporters detected.

### TECHNICAL FIELD

The invention relates generally to methods and compositions for predicting a change in physiological state of a subject based on molecular and architectural remodeling of cells, the extracellular matrix, and/or the tissue microenvironment.

### BACKGROUND

Many clinically important diseases involve changes to individual cells, the extracellular matrix, and/or the tissue microenvironment. The extracellular matrix is the collection of molecules surrounding the cells that provides support and connection in many bodily tissues. The tissue microenvironment is the collection of molecules secreted from and surrounding a population of cells that comprise tissue. Diseases that involve notable changes to the cells, extracellular matrix or tissue microenvironment include osteoarthritis, an inflammatory condition which is marked by the release of extracellular enzymes that degrade the extracellular matrix of cartilage and subject the underlying bone to damage and abrasion. It is also understood that tumors, and even certain infectious pathogens, advance within the body by causing the release of enzymes that release into the tumor microenvironment to break down the surrounding matrix to allow the disease to advance.

Treatment of such conditions is more effective when the condition is detected early. Early detection allows individuals to begin treatment before symptoms have progressed extensively. For example, arthritis can be treated most effectively if the underlying causes are addressed before the cartilage of a joint is extensively degraded. When conditions are detected before a condition has advanced significantly, the patient has the best chance of avoiding undue pain and experiencing a full recovery.

### SUMMARY

The invention provides compositions and methods for screening for, and/or diagnosing, conditions that involve architectural remodeling of individual cells, populations of cells, the extracellular matrix, and tissue microenvironment. Methods of the disclosure use activity sensors that, when delivered to an organ undergoing architectural remodeling in the course of disease progression, release detectable reporters from the activity sensors. The presence of those reporters in a sample taken from the subject is indicated of changes in the cellular and matrix architecture—as well as the tissue microenvironment—and can be correlated to the presence or stage of a disease or other physiological state of interest. For example, where the activity sensors include the substrates for a set of enzymes known to be involved in remodeling cellular architecture under a specific disease condition, detecting the reporters in the sample indicates the disease in the subject. Use of the activity sensors provides a tool for screening for, and/or diagnosing, conditions that involve the remodeling of cellular architecture, such as individual cells, the extracellular matrix, and tissue microenvironment including, for example, osteoarthritis, inflammation, fibrosis, cancer, and other conditions characterized by cellular remodeling.

Diseases that cause significant changes to cellular architecture include those that involve tissue inflammation or atrophy, traumatic injury, fibrosis and cancer, among others. In cancer, for example, cancerous cells excrete extracellular proteases into the tumor microenvironment that break down the highly-interconnected extracellular matrix to allow a tumor to appear and grow. Compositions of the disclosure can reveal the activity of those proteases before the tumor has even formed or grown to a threshold size that would be required for detection by imaging-based methods such as x-ray or tomography. Additionally, compositions and methods of the disclosure give a signal that indicates a rate of the change to cells, the extracellular matrix and tissue microenvironment. In another example, osteoarthritis involves the proteolytic decay of the cartilaginous extracellular matrix of joints, and the activity of proteases can be detected and measured using methods of the disclosure.

Because activity sensors of the disclosure release detectable reporters upon exposure to extracellular proteases or transmembrane proteases (those that span the cellular membrane and have an extracellular moiety), the activity sensors are useful for detecting remodeling of cellular architecture at the earliest stages of that remodeling and also for measuring the rate of the remodeling. Because a number of clinically significant diseases progress by cellular architectural remodeling, compositions of the disclosure are useful for early detection of those diseases. Because compositions of the disclosure are useful for detecting activity that is required before other stages of disease advancement such as tumor growth, compositions of the disclosure may be used to detect activity that is predictive of future disease stages such as tumor development.

In addition to detecting a state of disease, for example detecting presence of a tumor, compositions and methods of the disclosure allow detection of the rate of disease, such as rate of growth of a tumor. In other testing methods, a tissue sample from biopsy testing may detect the state of a disease or measure the degree of damage from the disease. However, the damage itself, such as the activity or rate of progression, may be detected by the present invention. As such, methods and compositions of the invention are used for measurements orthogonal to, or in conjunction with, imaging, biopsy, or blood tests, The invention provides an alternate approach to detect cellular architectural remodeling without the need for imaging, biopsy, or blood tests involving circulating markers

Because compositions and methods of the disclosure are useful for the very early detection of, and even for the early prediction of, a disease, compositions and methods provide for early disease screening, diagnosis, and intervention and allow clinicians to administer treatments at the earliest stages of disease. Because methods of the disclosure allow for early stage disease intervention and treatment, those treatments have an optimized chance for success, allowing more patients to go on to full recovery and avoiding painful symptoms and expensive hospital stays. Methods and compositions of the disclosure provide those benefits because they are useful for reporting the extent of cellular architectural remodeling within the tissue of a patient.

The extracellular matrix (ECM) is a dynamic structure present in tissues of organisms. The extracellular matrix is a network of proteins and sugars located around, and between, cells in every bodily tissue and provides physical support of the tissue. The extracellular matrix also helps maintain homeostasis in the tissue and is continuously undergoing changes due to enzymatic activity and cleavage of extracellular matrix components. Dysregulation of the extracellular matrix structure and composition is indicative of certain pathological conditions and disease progression.

For example, during malignant neoplastic progression, cells undergo genetic and epigenetic cancer-specific alterations that finally lead to a loss of tissue homeostasis and restructuring of the microenvironment. The invasion of cancer cells through connective tissue is part of metastasis. Although cell invasion is foremost a mechanical process, cancer research has focused largely on genetics/genomics. However, the structural and biomechanical properties of extracellular matrix and surrounding cells such as endothelial cells influence cancer cell motility and invasion. Thus, the tissue microenvironment is one critical determinant of the migration strategy and the efficiency of cancer cell invasion.

In normal conditions, such as for healthy adult tissues, certain molecules are expressed in the cellular and extracellular environment at low levels or not at all. However, certain pathologies are associated with persistent expression of molecules in the extracellular environment. As such, medically significant hallmarks of disease are detectable in the extracellular environment. Because architectural remodeling occurs at the extracellular and/or cellular transmembrane level, the disease hallmarks are detectable at the level of cells, populations of cells, the extracellular matrix and tissue microenvironment, before they are detectable in other areas of the body, such as the blood. Due to the different extracellular matrix or tissue microenvironment compositions in different areas of the body, the invention also allows for localization or detection of the site of the disease hallmarks. For example, activity happening in adipose tissue is different from and detectable independent of activity happening in the liver. As such, detecting disease hallmarks in cells, the extracellular matrix or tissue microenvironment during initial stages of the disease or in a pre-disease state is possible.

The present invention provides methods of detecting the disease hallmarks in the extracellular matrix or tissue microenvironment. Due to the nature of the disease hallmarks, methods of the present invention allow for detection of disease hallmarks earlier than other available testing methods. For example, other detection methods include liquid biopsy, the typical diagnosis method for early detection of inflammatory and atrophic diseases, such as cancer. In liquid biopsy, biomarkers for a particular disease may be detected from a blood sample. However, the present invention detects enzymatic activity associated with cellular architectural remodeling, and thus allows for detection before the pathological condition is detectable in the blood.

In certain aspects, the invention provides methods for detecting or measuring changes in cells, populations of cells, the extracellular matrix and tissue microenvironment. The method include administering to a body of a subject a plurality of activity sensors that release detectable reporters when cleaved by enzymes that remodel the extracellular matrix or tissue microenvironment under a specific condition, detecting the detectable reporters in a sample collected from the subject, and correlating the detectable reporters to the specific condition in the subject. The methods may include quantifying the detectable reporters in the sample and reporting a rate of change in the architectural remodeling in the cells or tissue of the subject. The methods may optionally include correlating a quantity of the detectable reporter detected to an extent of remodeling of the extracellular matrix or tissue microenvironment in the subject. Any suitable condition or disease may be interrogated including, for example, a proliferative disorder or cancer. When the disease is a cancer, the method may include predicting the appearance of a tumor in the subject. Preferably, the method includes describing the extent of the remodeling and predicting the tumor before a tumor is able to be seen via an imaging method or via pathology, staining, etc.

In some embodiments, the method includes reporting an onset of the disease in the subject. The method may be useful for diseases characterized by either inflammation or atrophy. For example, the disease may be cancer, arthritis, traumatic injury, or muscle injury. In certain embodiments, the disease is cancer and architectural remodeling of the extracellular matrix or tissue microenvironment comprises development of a tumor. In other embodiments, the disease is arthritis and the architectural remodeling involves a loss of cartilage. The detectable reporters may be detected by any suitable assay including, for example, mass spectrometry or an immunoassay. The method may be used for longitudinal or repeated monitoring of changes in disease status in the subject after a treatment has been administered.

Preferably, each of the plurality of activity sensors includes detectable reporters specific to one enzyme. The plurality of activity sensors may collectively include detectable reporters specific to: a plurality of enzymes that exhibit the disease-specific architectural remodeling of the extracellular matrix and/or tissue microenvironment, and at least one enzymes that does not exhibit disease-specific remodeling of extracellular matrix and/or tissue microenvironment. Each activity sensor may include a carrier comprising one or a plurality of molecular subunits and the plurality of reporters, each linked to the carrier by a cleavable linker containing a cleavage site of the one or more extracellular matrix remodeling enzymes. The carrier may be provided as a 20 to 60 kDa scaffold of linked polyethylene glycol (PEG) subunits. Each reporter and cleavable linker may include a polypeptide susceptible to cleavage by a protease known to be associated with a specific disease or condition associated with cellular architectural remodeling.

Any suitable detection or testing methods may be used to detect the liberated reporters. In certain embodiments, the liberated reporters are detected by an enzyme immunoassay. In other embodiments, testing methods used to detect the liberated reporters include mass spectrometry, ELISA, and chromatography. In an embodiment, the physiological state of the subject may be monitored after a course of treatment has been prescribed. As an example, the invention may be used to predict that the subject is in a pre-disease state. A therapeutic treatment may be administered to the subject for a prescribed time period. The invention may then be used to detect enzymatic activity after the prescribed time period. Effectiveness of the therapeutic treatment may be determined based upon a comparison of the enzymatic activity before and after the therapeutic treatment, and can be repeated to enable longitudinal monitoring of changes in disease in the subject over time.

In related aspects, the invention provides a composition for detecting, monitoring, or measuring a rate of remodeling of architectural changes in the extracellular matrix and tissue microenvironment. The composition includes a plurality of activity sensors, each activity sensor including detectable reporters susceptible to an enzyme that exhibits disease-specific architectural remodeling of extracellular matrix and tissue microenvironment. The activity sensors may optionally include “control” reporters susceptible to cleavage by an enzyme not know to exhibit disease-specific architectural remodeling of extracellular matrix and tissue microenvironment. In an embodiment, the activity sensor comprises a carrier and a plurality of reporters. The carrier comprises one or a plurality of molecular subunits. Each of the plurality of reporters is linked to the carrier by a cleavable linker containing a cleavage site of the one or more architectural remodeling enzymes. One or more reporters are liberated upon cleavage by the one or more architectural remodeling enzymes, thereby reporting enzymatic activity. The carrier may include a 20 to 60 kDa (e.g., approximately 40 kDa) poly ethylene glycol (PEG) scaffold of covalently linked PEG subunits. Further, each reporter and cleavable linker comprises a polypeptide susceptible to cleavage by a protease known to be associated with a specific disease or condition associated with architectural remodeling of the extracellular matrix or tissue microenvironment.

Aspects of the disclosure provide a method of detecting changes in extracellular matrix or tissue microenvironment. The method includes administering to a subject a composition comprising a plurality of activity sensors that release detectable reporters under conditions indicative of remodeling of an architecture of extracellular matrix or tissue microenvironment; detecting the detectable reporters in a sample collected from the subject; and correlating the detectable reporters to a specific condition in the subject. The composition may detect one or more of connective tissue growth factor molecules (e.g., CTGF or CCN2), fibrin or blood clot formation, osteopontin or bone resorption. The composition includes a plurality of activity sensors, each activity sensor including detectable reporters susceptible to an enzyme that exhibits disease-specific remodeling of extracellular matrix or tissue microenvironment. Those enzymes may be extracellular, intracellular, or membrane-bound, but exhibit activity that results in changes to the extracellular matrix or tissue microenvironment. The activity sensors may optionally include “control” reporters susceptible to cleavage by an enzyme not known to exhibit disease-specific remodeling of extracellular matrix or tissue microenvironment. In an embodiment, the activity sensor comprises a carrier and a plurality of reporters. The carrier comprises one or a plurality of molecular subunits. Each of the plurality of reporters is linked to the carrier by a cleavable linker containing a cleavage site of the one or more extracellular matrix or tissue microenvironment remodeling enzymes. One or more reporters are liberated upon cleavage by the one or more extracellular matrix or tissue microenvironment remodeling enzymes, thereby reporting enzymatic activity. The carrier may include a 30 to 60 kDa (e.g., approximately 40 kDa) poly ethylene glycol (PEG) scaffold of covalently linked PEG subunits. Further, each reporter and cleavable linker comprises a polypeptide susceptible to cleavage by enzyme known to be associated with a specific disease or condition associated with changes to extracellular matrix or tissue microenvironment.

### BRIEF DESCRIPTION OF THE DRAWINGS

FIG. 1 shows a method for detecting changes to the extracellular matrix where those changes may be predictive or characteristic of disease or physiological state of interest.

FIG. 2 shows an activity sensor useful in methods of the disclosure.

FIG. 3 illustrates use of a composition for describing remodeling of extracellular matrix.

FIG. 4 shows a composition for describing changes to the extracellular matrix.

FIG. 5 illustrates a carrier useful in certain embodiments.

FIG. 6 illustrates determining elements of a composition.

FIG. 7 shows cleavage by extracellular proteases to release detectable reporters.

FIG. 8 illustrates an assay result by which one detects detectable reporters.

### DETAILED DESCRIPTION

The invention provides compositions and methods for screening for, and/or diagnosing, conditions that involve architectural remodeling of individual cells, populations of cells, the extracellular matrix, and tissue microenvironment. Methods of the disclosure use activity sensors that, when delivered to an organ undergoing architectural remodeling in the course of disease progression, release detectable reporters from the activity sensors. The presence of those reporters in a sample taken from the subject is indicated of changes in the cellular and matrix architecture. When the activity sensors include the substrates for a set of enzymes known to be involved in remodeling cellular architecture under a specific disease condition, detecting the reporters in the sample indicates the disease in the subject. Diseases that cause significant changes to cellular architecture include those that involve tissue inflammation or atrophy, traumatic injury, fibrosis and cancer, among others. In cancer, for example, cancerous cells excrete extracellular proteases into the tumor microenvironment that break down the highly-interconnected extracellular matrix to allow a tumor to appear and grow. Compositions of the disclosure can reveal the activity of those proteases before the tumor has even formed or grown to a threshold size that would be required for detection by imaging-based methods such as x-ray or tomography. Because activity sensors of the disclosure release detectable reporters upon exposure to extracellular proteases or transmembrane proteases that span the cellular membrane and have an extracellular moiety, the activity sensors are useful for detecting remodeling of cellular architecture at the earliest stages of that remodeling and also for measuring the rate of the remodeling. Because a number of clinically significant diseases progress by cellular architectural remodeling, compositions of the disclosure are useful for early detection of those diseases. Because compositions of the disclosure are useful for detecting activity that is required before other stages of disease advancement such as tumor growth, compositions of the disclosure may be used to detect activity that is predictive of future disease stages such as tumor development.

Because compositions and methods of the disclosure are useful for the very early detection of, and even for the early prediction of, a disease, compositions and methods provide for early disease screening, diagnosis, and intervention and allow clinicians to administer treatments at the earliest stages of disease. Because methods of the disclosure allow for early stage disease intervention and treatment, those treatments have an optimized chance for success, allowing more patients to go on to full recovery and avoiding painful symptoms and expensive hospital stays. Methods and compositions of the disclosure provide those benefits because they are useful for reporting the extent of cellular architectural remodeling within the tissue of a patient.

The extracellular matrix (ECM) is a dynamic structure present in tissues of organisms. The extracellular matrix is a network of proteins and sugars located around, and between, cells in every bodily tissue and provides physical support of the tissue. The extracellular matrix also helps maintain homeostasis in the tissue and is continuously undergoing changes due to enzymatic activity and cleavage of extracellular matrix components. Dysregulation of the extracellular matrix structure and composition is indicative of certain pathological conditions and disease progression. For example, during malignant neoplastic progression, cells undergo genetic and epigenetic cancer-specific alterations that finally lead to a loss of tissue homeostasis and restructuring of the microenvironment. The invasion of cancer cells through connective tissue is part of metastasis. Although cell invasion is foremost a mechanical process, cancer research has focused largely on genetics/genomics. However, the structural and biomechanical properties of extracellular matrix and surrounding cells such as endothelial cells influence cancer cell motility and invasion. Thus, the tissue microenvironment is one critical determinant of the migration strategy and the efficiency of cancer cell invasion.

In normal conditions, such as for healthy adult tissues, certain molecules are expressed in the cellular and extracellular environment at low levels or not at all. However, certain pathologies are associated with persistent expression of molecules in the extracellular environment. As such, medically significant hallmarks of disease are detectable in the extracellular environment. Because architectural remodeling occurs at the extracellular and/or cellular transmembrane level, the disease hallmarks are detectable at the level of cells, populations of cells, the extracellular matrix and tissue microenvironment, before they are detectable in other areas of the body, such as the blood. Due to the different extracellular matrix or tissue microenvironment compositions in different areas of the body, the invention also allows for localization or detection of the site of the disease hallmarks. For example, activity happening in adipose tissue is different from and detectable independent of activity happening in the liver. As such, detecting disease hallmarks in cells, the extracellular matrix or tissue microenvironment during initial stages of the disease or in a pre-disease state is possible.

The activity sensors of the present invention detect signals generated in an organism from activity, which may be enzymatic activity. In some instances, the activity sensors detect certain non-enzymatic responses. As an example, the activity may be related to conformation, and the activity sensors may detect acid, sheer, or competitive binding events. For example, the activity sensors may detect results of a mechanical event, such as leakage from red blood cells getting squeezed during cellular damage or membrane damage. As an example, a peptide may assume different conformations, or may fold differently, based on a pH change in the body.

Signals detected by the activity sensors may also indicate secondary interactions happening within the organism. The secondary interactions may be the result of events happening locally biologically. For example, signal A may indicate the activity that results in cells being killed within the body, while signal B indicates the results from the cells being killed. The activity sensors may detect signal A on its own, signal B on its own, and signals A and B together. The activity sensors provide greater sensitivity and specificity, as signals A and B may be detected together. Detection may indicate that signals A and B are happening within the same area locally in the body or that signals A and B are happening in different locations.

The signals detected by the present invention may indicate a state of disease, for example detecting presence of a tumor. Compositions and methods of the disclosure also allow detection of the rate of disease, such as rate of growth of a tumor. In other testing methods, a tissue sample from biopsy testing may detect the state of a disease or measure the degree of damage from the disease. However, the damage itself, such as the activity or rate of progression, may be detected by the present invention. As such, methods and compositions of the invention are used for measurements orthogonal to imaging.

For example, a number of subjects may be diagnosed with tumors. Checkpoint blockade drugs may be administered to those subjects. After administration of the checkpoint blockade drugs, the tumors may grow in size. For some of the subjects, the tumor growth may be due to ineffective treatment. For other subjects, the tumor growth may be due to an influx of immune cells to the tumor. The present invention detects the activity of the immune cell influx and provides an indication that the subject is responding to treatment even though the tumor is initially growing in size. Traditional detection methods such as imaging would merely indicate that the tumors are growing and thus the subjects are not responding to treatment.

As another example, activity sensors of the invention may detect tissue that is fibrotic and indicative of a disease such as nonalcoholic steatohepatitis (NASH). However, the activity sensors of the invention may further detect enzymatic activity happening within that fibrotic tissue. As such, the activity sensors would detect that the fibrotic tissue is still active tissue and thus would not be considered “burnt out NASH” or inactive fibrotic tissue. Imaging and conventional testing and detection methods do not provide detection of such activity.

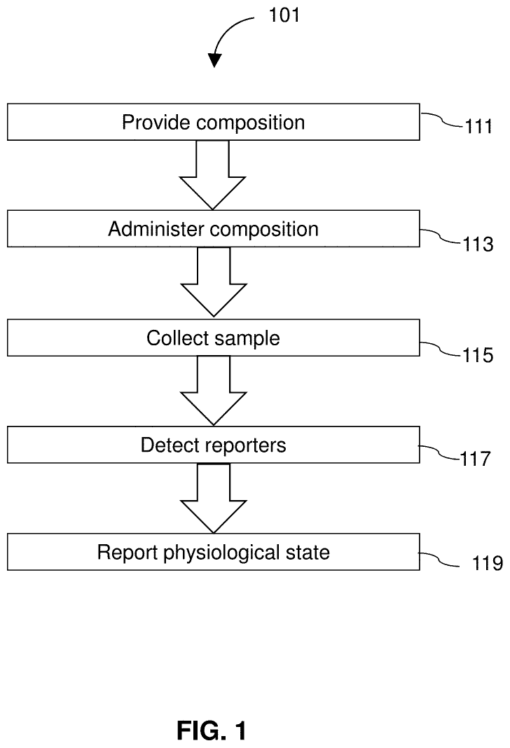

FIG. 1 shows a method 101 for detecting changes to the extracellular matrix where those changes may be predictive or characteristic of disease or physiological state of interest. The method 101 includes providing 111 a composition that includes a plurality of activity sensors. Providing 111 the composition may include designing or determining the specific activity sensors to include based on profiling activity of one or more proteases that are active under the physiological state of interest. The composition is administered 113 to a subject. Any suitable introduction method may be used. For example, the composition may be introduced to a subject by intravenous injection.

Where a patient is affected by a condition that involves remodeling of the extracellular matrix of tissue, the activity sensors in that tissue encounter proteases that participate in the remodeling. The protease cleaves and releases detectable reporters from the activity sensors. A sample is collected 115 from the patient and assayed 117 for the presence of the released detectable reporters. Where the results of the assay indicate the presence of cleaved detectable reporter in the sample, the method 101 may be used to provide 119 a report that describes a physiological state of the tissue such as, for example, reporting the onset or stage of the disease affecting the tissue.

Methods of the disclosure use compositions that include nanoparticle-scale activity sensors that release detectable reporters when acted upon by extracellular proteases, the presence of which is characteristic of the physiological state (e.g., disease) of interest.

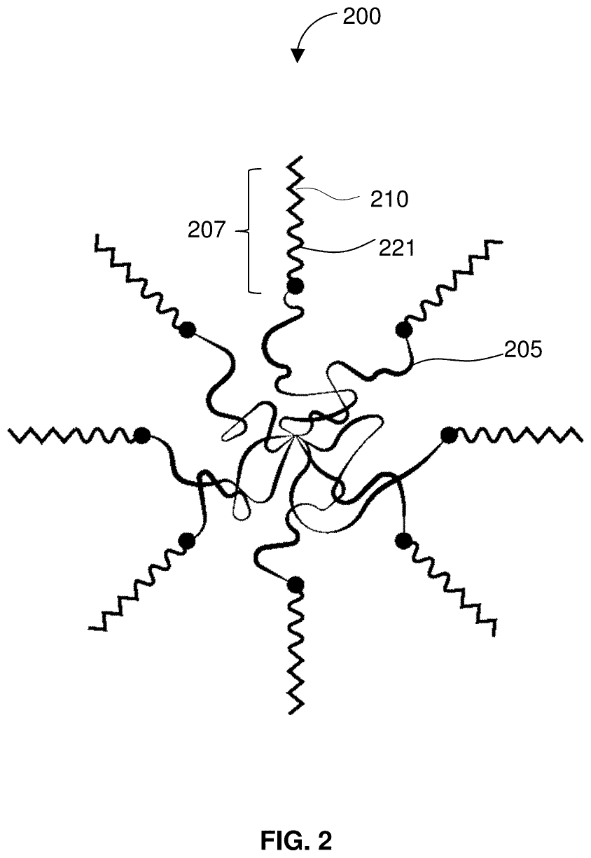

FIG. 2 shows an activity sensor 200 useful in methods of the disclosure. The activity sensor 200 includes one or a plurality of detectable reporters 210. Any suitable detectable reporter may be provided so long as it is released from the activity sensor 200 and is then detectable in a sample from the patient when the activity sensor 200 encounters one or more enzymes that participate in remodeling of extracellular matrix. In certain embodiments, the detectable reporters 210 are provided by polypeptides 207 that may be linked to a suitable carrier 205, such as a molecular scaffold comprising one or a plurality of biocompatible molecular subunits. In an aspect, the carrier 205 comprises a 30 to 40 kDa poly ethylene glycol (PEG) scaffold of covalently linked PEG subunits. Further, each reporter and cleavable linker may comprise a polypeptide susceptible to cleavage by a protease known to be associated with a specific disease or condition associated with extracellular matrix remodeling. In preferred embodiments, the polypeptides 207 each include a substrate and cleavage site 221 for an extracellular protease that is active under the physiological condition of interest.

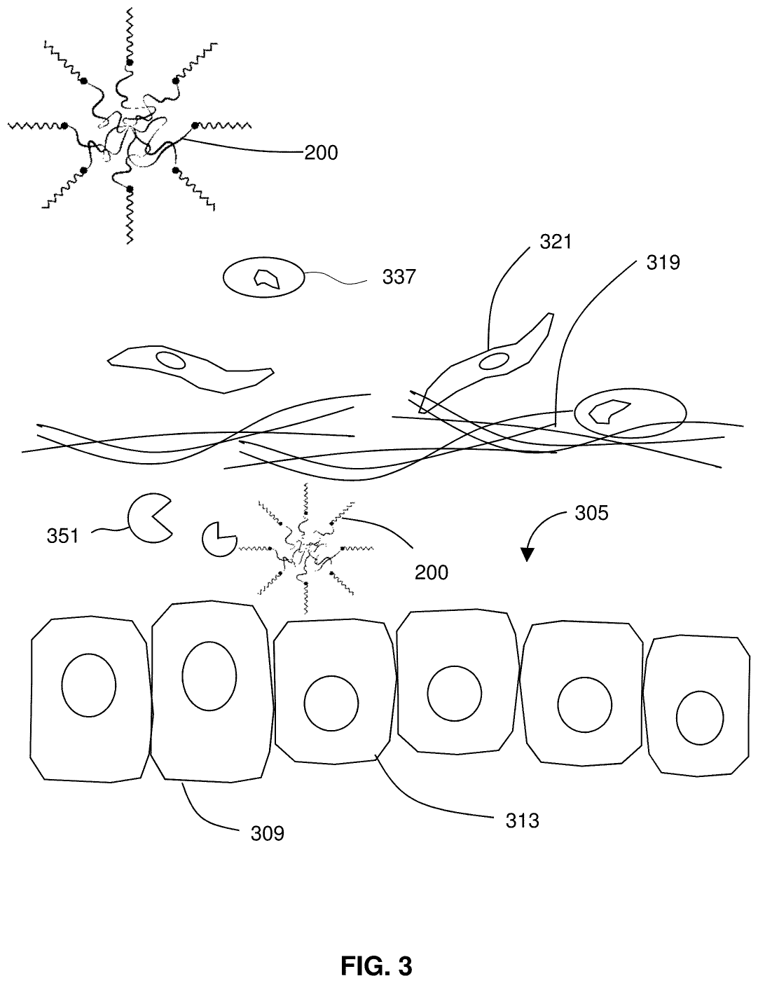

FIG. 3 illustrates use of a composition of the disclosure for detecting, reporting, and describing remodeling of extracellular matrix 319 by extracellular proteases 351 at a site within tissue of a subject. In the depicted embodiment, the extracellular matrix 319 overlies the basement membrane 305. Here, the basement membrane 305 includes a plurality of epithelial cells 313 and one tumor prone epithelial cell 309. The extracellular matrix is also populated with a mix of cells such as fibroblasts 321 and macrophages 337. The tumor-prone epithelial cell 309 may begin to make and secrete extracellular proteases 351 before any significant proliferation.

Absent the activity of the extracellular proteases 351, the extracellular matrix 319 physically constrains proliferation of the cells. The extracellular proteases 351 cleave proteins of the extracellular matrix 319 allowing the tumor-prone cell 309 to begin development into a tumor. A composition of the disclosure that includes a plurality of the activity sensors 200 is administered to the subject. The activity sensors 200 encounter the extracellular proteases 351 within the extracellular matrix 319. The proteases cleave the peptides 207 and release the detectable reporters. In the depicted embodiment, the released detectable reporters will collect into the bloodstream after which they are subject to glomerular filtration and release in urine.

Thus according to method 101, after administration 213 of the composition (e.g., about one or a couple of hours after) a sample is collected 115 from the subject. In an example, the sample collected is a urine sample. The sample is analyzed to detect 117 reporters released from the activity sensors. A set of proteases active together in tissue at any given time provides a sensitive marker along a continuum of health and may indicate a disease and a stage of the disease.

As shown in FIG. 3, the activity sensors 200 are useful for detecting changes in the extracellular matrix 319 in tissue of a subject. The extracellular matrix is a dynamic structure present in all tissues. The extracellular matrix 319 is a network of proteins and sugars located around and between cells in every bodily tissue. In particular, the extracellular matrix is composed of proteoglycans and fibrous proteins, such as collagens, elastins, fibronectins, and laminins. The extracellular matrix 319 is responsible for physical support of the tissue, as well as tissue homeostasis, or maintenance, of the internal steady state within the tissue. Due to the role of the extracellular matrix in maintaining tissue homeostasis, the extracellular matrix is continuously undergoing changes due to enzymatic activity and cleavage of extracellular matrix components. Extracellular matrix remodeling happens through cellular synthesis, degradation, reassembly, and chemical modification.

Dysregulation of proteases 351 is genetically selected during cancer and that distinct members of each group may serve similar roles in tumors. See Sjöblom, 2006, Science 314:268-274, incorporated by reference. The extracellular matrix 319 constantly undergoes remodeling during normal conditions. However, dysregulation of the extracellular matrix structure and composition is commonly associated with disease. In particular, pathological conditions and disease progression are associated with dysregulated extracellular matrix remodeling. Hallmarks of disease that are medically significant, such as activity by extracellular proteases 351, are detectable in the extracellular matrix, and activity by extracellular proteases 351 may be detectable before other symptoms are detectable in other areas of the body, such as the blood. Thus, it is possible to detect disease hallmarks in the extracellular matrix during initial stages of the disease or in a pre-disease state.

Several hallmarks of disease are associated with changes in the extracellular matrix. In normal conditions, such as for healthy adult tissues, certain molecules are expressed at low levels or not at all. Certain pathologies are associated with persistent expression of molecules in the extracellular matrix, characteristic of dysregulated inflammation, tissue deposition, and angiogenesis. For example, abnormal stiffness and extracellular matrix deposition are characteristic of fibrosis and cancer, while excessive extracellular matrix degradation is characteristic of osteoarthritis.

As a specific example, connective tissue growth factor molecules, such as CTGF and CCN2, are markers of disease. Connective tissue growth factor molecules are typically directed to cell proliferation and induce angiogenesis and new tissue synthesis during tissue repair. Overexpression of the connective tissue growth factor molecules is characteristic of fibrosis, such as renal, liver, and cardiac fibrosis, diabetic neuropathy, and solid tumors, such as pancreatic cancer.

As another specific example, fibrin molecules typically are directed to blood clot formation and act as a scaffold for new tissue synthesis during tissue repair. Persistent expression of fibrin is characteristic of fibrosis, such as renal, lung, and dystrophic muscle fibrosis, and solid tumors, such as small cell carcinoma of the lung and renal cell carcinoma.

Another specific example relates to osteopontin, which initiates bone resorption by osteoclasts and is a pro-inflammatory mediator during tissue repair. However, solid tumors, such as breast cancer, prostate cancer, and pancreatic ductal adenocarcinoma, are characterized by persistent expression of osteopontin. Persistent osteopontin expression is also characteristic of chronic inflammation, such as rheumatoid arthritis, multiple sclerosis, and systemic lupus erythematosus. Any such disease condition associated with dysregulated (i.e., increased or decreased) levels of activity by extracellular proteases may be interrogated using methods and compositions of the disclosure.

Secondary activity occurring in extracellular matrix or a tissue microenvironment may also be detected by methods and compositions of the invention. For example, blood clots cause a physical response in the body. The physical response caused by the blood clots is secondary activity that produces a detectable signal. For example, the composition may be used for detection of binding moieties in enzymes and fibrin clot formation, thus indicating binding or incorporation into the ECM depending on cleavage. Compositions and methods of the invention may be used to measure co-location of activity.

Other examples of secondary activity include any action or interaction in the body resulting from administration of the composition, such as inducing an immune response, binding events, or steric or chemical responses to the composition.

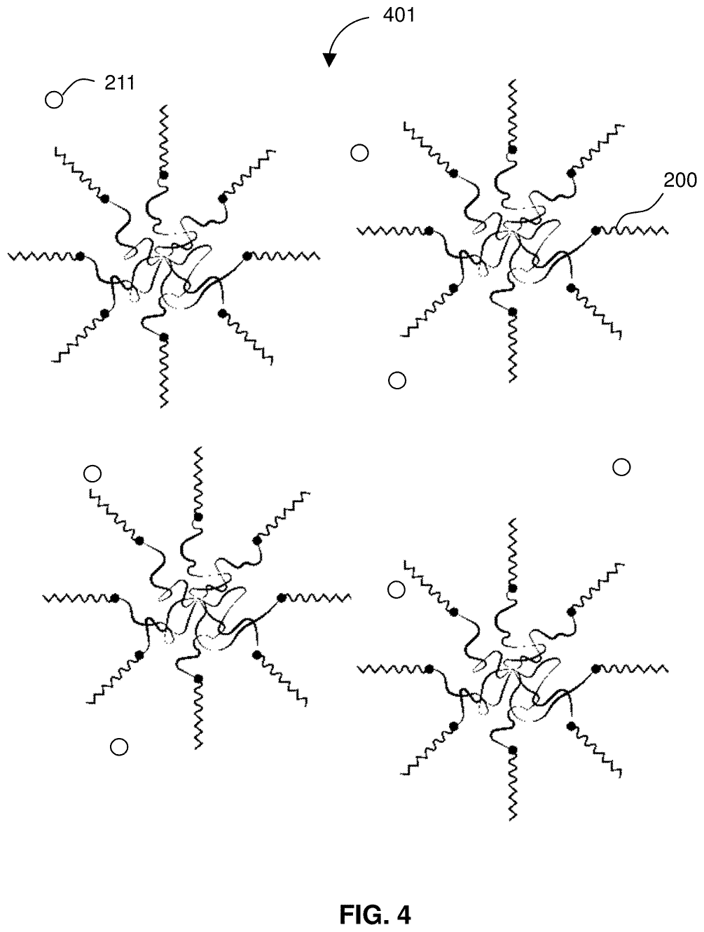

FIG. 4 shows a composition 401 for detecting and describing changes to the extracellular matrix. The composition 401 includes a plurality of activity sensors 200. Each activity sensor 200 includes one or more detectable reporter 210 susceptible to cleavage and release by a specific extracellular protease 351. The detectable reporters 210 may be provided by polypeptides 207 linked to a suitable carrier 205, such as a molecular scaffold comprising one or a plurality of biocompatible molecular subunits. Optionally, the activity sensors 200 are provided in a pharmaceutically acceptable carrier 211 such as an aqueous suspension, ointment, cream, or gel. Any suitable structure may be used for the carrier 205. In preferred embodiments, the carrier 205 includes a biocompatible polymer such as polyethylene glycol, polylactic acid, lipids, carbohydrates, or other such structures.

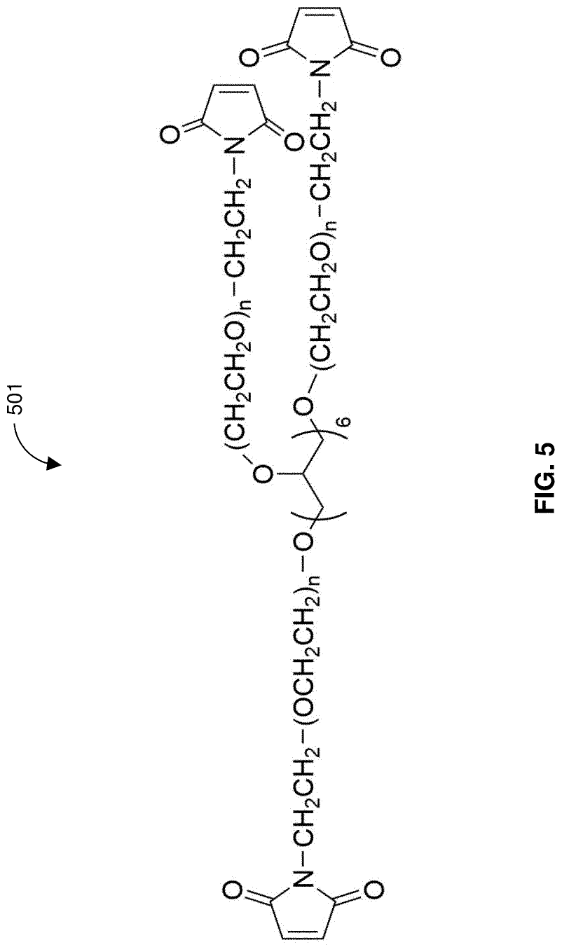

FIG. 5 illustrates a carrier 501 useful in certain embodiments. In some embodiments, the carrier comprises a polymer with a mass between about twenty and fifty kDa. The carrier may include a multi-arm polyethylene glycol (PEG) scaffold to which each reporter is covalently linked. The depicted carrier includes an 8-arm, 40 kDa PEG scaffold. Thus in certain embodiments, the disclosure provides a pro-analyte that includes an activity sensor in which the detectable reporter is linked to a carrier comprising, for example, one or more polymeric subunits. For example, the carrier may be a multi-arm PEG scaffold (e.g., 8-arm PEG at about 40 kDa).

Preferably, the composition 401 includes a plurality of activity sensors 200, each of which includes a plurality of detectable reporters 210 such that the composition releases the detectable reporters in the presence of a plurality of extracellular proteases 351. Optionally, each activity sensor has a number (e.g., 8) of substantially identical polypeptide side chains 207 that, when cleaved by a protease 351, release a portion of the polypeptide side chain as the detectable reporter 210. The composition 401 may release detectable reporters for a plurality of different proteases 351 by having numerous activity sensors, each with peptide side chains 207 specific for one protease 351. Preferably, the composition 401 releases the detectable reporters 210 upon encountering a plurality of the proteases 351. The composition 401 may also include detectable reporters specific to enzymes that are not specific to the disease state. That is, the composition may include certain activity sensors as a “control”, or to report a baseline activity level for one or more enzymes not specifically associated with the disease. When the proteases 351 cleave the activity sensors to release the detectable reporters, detection of those reporters in samples from the body indicates the presence of remodeling extracellular matrix of the tissue. Of note, many diseases involve mechanisms of action by which enzyme activity is dysregulated prior to the appearance of other symptoms. For example, neoplastic cells will release extracellular tissue remodeling enzymes to cleave the extracellular matrix before the cells proliferate into a detectable tumor and before those tumor cells release a level of tumor DNA fragments that can be detectable by liquid biopsy. Accordingly, the precise detection of extracellular proteases at a specific site is indicative of an important physiological state.

In some embodiments, the composition 401 includes at least one activity sensor with a substrate for an enzyme indicative of healthy tissue, thereby indicating a baseline enzyme activity level of the subject. Similarly, the composition may include activity sensors with substrates for enzymes that are indicative of comorbidities of that specific disease or condition, and the present invention provides a therapeutic profile for the subject.

Methods of the disclosure provide for determining 111 a set of activity sensors to include in a composition 401, i.e., for determine what set of proteases of interest should be addressable or addressed using a composition of the disclosure and thus selecting the relevant polypeptides 207 to include for the detectable reporters. For each protease that it is intended to interrogate, one specific activity sensor 200 may be included, that activity sensor preferably linked to a plurality (e.g., 8) of polypeptides 207 that include a cleavage site 221, e.g., a scissile bond susceptible to cleavage by that protease. The activity sensors 200 optionally include a pro-analyte moiety 215 that is removed by the process in the body. Using such activity sensors 200, methods 101 include detecting 117 the detectable reporters released from the activity sensors at the target site to thereby sense remodeling of extracellular matrix.

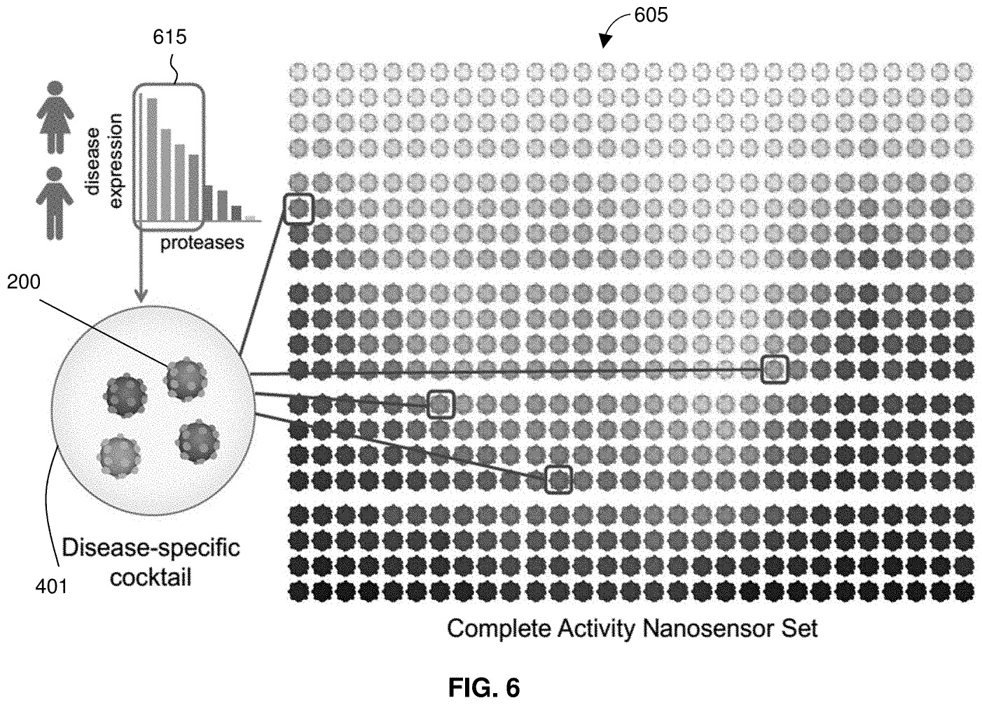

FIG. 6 illustrates determining 111 elements of a composition 401 according to the disclosure. Preferably, a composition 1101 is made by drawing from a library 605 that contains a complete set of activity sensors 200. In certain embodiments, each activity sensor 200 within the library 605 includes a plurality of identical polypeptide side chains. Each library entry may be represented by an arbitrary number (e.g., thousands) of copies that may be identical or essentially or functionally identical. As shown, the library 605 has 580 unique activity sensors 200. Each one may be present in thousands of copies stored in a suitable container. For example, in some embodiments, the hundreds or thousands of copies of each library member is stored in its own container such as a centrifuge tube such as the 1.5 mL micro-centrifuge tube sold under the trademark EPPENDORF FLEX-TUBES by Eppendorf, Inc. (Enfield, Conn.). For any given biological or physiological condition of interest, a profile 615 of relevant proteases is developed or obtained.

The profile 615 includes extracellular proteases 351 that are differentially expressed under the physiological state of interest, and such enzymes are preferably included in the profile 615. However, the profile 615 may also include other enzymes, such as one or more control enzymes that are assumed to be constitutively expressed and active in the tissue or bodily compartment of interest. The control enzyme may give a background activity level against which to calibrate or normalize the activity levels of the disease-associated enzymes. Additionally, the profile may include one or more enzymes that are understood to be specific for a frequent or likely comorbidity of the physiological state of interest. The activity sensors 200 specific to the extracellular proteases 351 in the profile 615 are composed together to form a composition 401 of the disclosure. Thus methods of the disclosure provide for designing compositions 401 that include activity sensors 200 in which each activity sensor 200 includes a plurality of detectable reporters 210 such that the composition 401 releases the detectable reporters 210 in the presence of a plurality of cognate extracellular protease 351. Those extracellular proteases 351 include one or more of the extracellular protease 351 that exhibit elevated activity in association with a disease state.

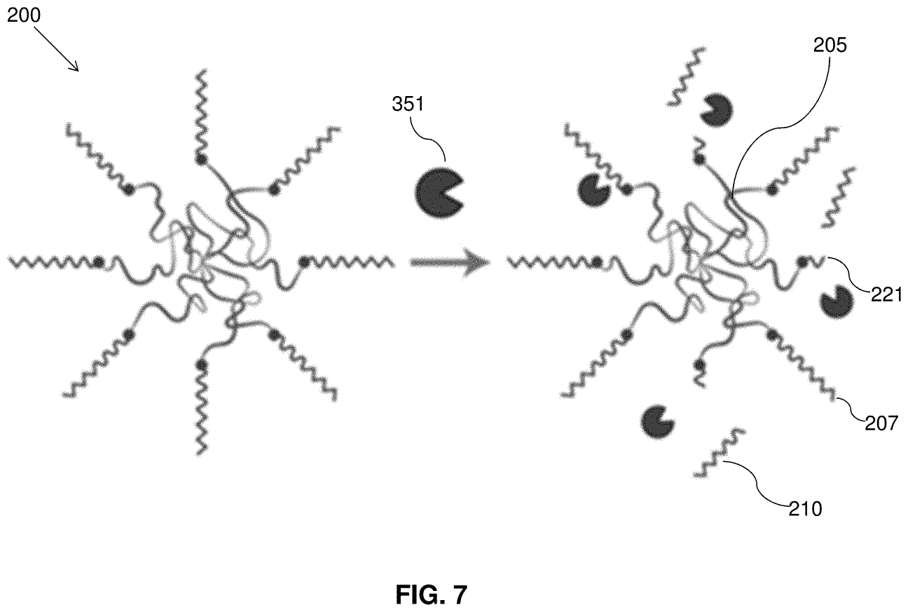

FIG. 7 shows cleavage of the activity sensors 200 by the extracellular proteases 251 to release the detectable reporters 210. Because the activity sensors are designed to include the substrates 221 for the respective protease 351 in the peptide chains 307, when the activity sensors encounter the proteases 351, the detectable reporters are released. Due to their small size, the detectable reporters 210 are free to diffuse through tissue and they collect in blood where they circulate until clearance in urine by glomerular filtration. By such filtration, the patient releases the detectable reporters 210 into a sample that can be collected 115 and analyzed 117.

The sample may be analyzed 117 by any suitable method or assay. Suitable bodily samples, depending on the nature of localization of the sensors 200, include blood, urine, sweat, lymph, a biopsy, fine needle aspirate, exhaled breath, a swab (e.g., cheek swab), tears, mucous, cerebrospinal fluid, tissue sample, resected tumor sample, hair or nail clipping, cartilage excision, synovial fluid (e.g., from a joint affected or suspected to be affected by osteoarthritis), or any other suitable sample known in the art. Suitable assays for detecting the detectable reporters include, for example, enzyme-linked immunosorbent assays, other immunoblotting assay, mass spectrometry, secondary ion mass spectrometry, gel electrophoresis, two-dimensional electrophoresis, chromatography, HPLC, bead capture and separation (e.g., using magnetic beads that bind to the detectable reporters), or any other assay. Where the analytes each have a unique mass by virtue of the design of the polypeptide sequence, mass spectrometry may be performed on the urine sample to reveal the presence or absence of mass spectra signifying the presence or absence of the physiological state within a patient.

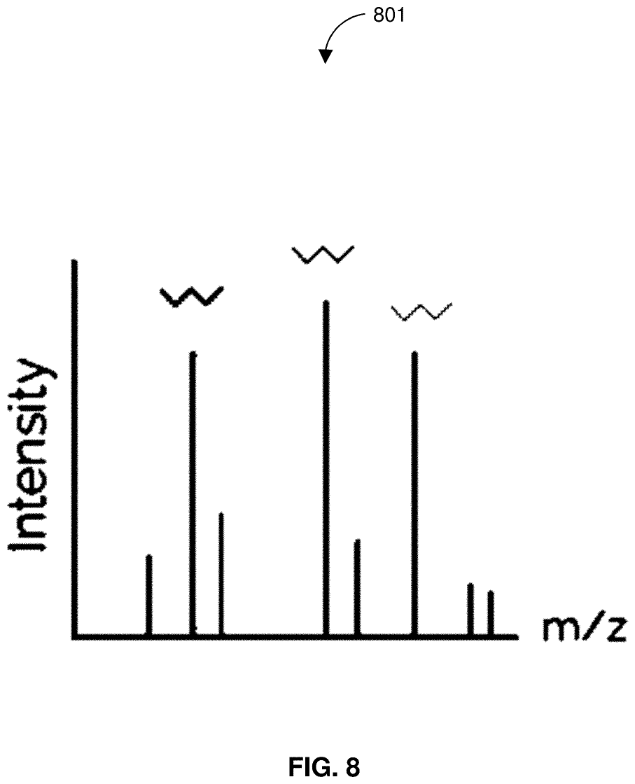

FIG. 8 illustrates an assay result 801 by which one detects 117 detectable reporters 210. In the depicted embodiment, the assay result 801 is a mass spectra as may be obtained from a sample from a patient. The sawtooth lines represent the detectable reporters 210, and that each has a distinguishing mass to charge (m/z) ratio. The presence of the indicated peaks on the mass spectra indicates that the proteases were present in the liver and cleaved the reporters.

The activity sensors 200 of the present invention detect dysregulated proteases 351 associated with disease and remodeling of extracellular matrix. Activity sensors 200 may be formulated from any suitable material and preferably are PEG and mass-barcoded protease substrates. The activity sensors each comprise a plurality of detectable reporters. In preferred embodiments, the reporters are peptide substrates designed to be cleaved by specific proteases.

Embodiments of the present invention are directed to methods of predicting a physiological state of a subject. A composition comprising activity sensors 200 is introduced into a body of a subject. The activity sensors comprise a plurality of reporters susceptible to cleavage by one or more extracellular protease 351 that are predictive of a physiological state. A sample is collected 115 from the subject, and liberated reporters 210 are detected in the sample. An onset of the physiological state of the subject may be diagnosed based on the liberated reporters 210 detected. The physiological state of the subject may be a disease characterized by inflammation or atrophy.

Additionally, because the activity sensors 200 provide an excess number of substrates for the enzymatic cleavage, the presence of reporters or detectable analytes in a sample from the body may be measured quantitatively to give a measure of rate of activity of the proteases. Collectively, the rates of activity of the enzymes may serve as an instantaneous measure of rate of progression of the disease.

Any suitable method may be used for detection and analysis. For example, methods of detection include mass spectrometry, chromatography, volatile organic compounds (VOC), enzyme immunoassay, such as enzyme-linked immunosorbent assay (ELISA), imaging, such as magnetic imaging, breath analyzer, and single molecule, paper diagnostic, nucleic acid coding, imaging, and control probes. In a preferred embodiment, the method of detection is enzyme immunoassay. For example, an immunoassay using antibodies for the reporters or detectable analytes may be a preferred detection and analysis method. In a preferred embodiment, the sample is analyzed by mass spectrometry (which assays for a mass to charge ratio of polypeptides cleaved from the activity sensors).

In certain embodiments, the result of detection and analysis provides a physiological profile of the subject, which may then be provided 119 in a report. For example, results may indicate a stage and/or rate of progression of a disease in the tissue (e.g., peaks in mass spectra). The report may describe the therapeutic profile of a subject and may further indicate a physiological condition of the subject. The report may be used for diagnosis, monitoring, and/or treatment of the subject.

Thus methods and compositions of the invention are useful for predicting a physiological state of a subject. A composition 401 comprising activity sensors 200 is introduced into a body of a subject. The activity sensors comprise a plurality of detectable reporters 210 susceptible to cleavage by one or more extracellular matrix remodeling enzymes 351 that are predictive of a physiological state. A sample is collected 115 from the subject, and liberated reporters are detected in the sample. Methods 101 and compositions of the disclosure may be used with any suitable subject. For example, the subject may be a human subject. In certain embodiments, methods and composition of the invention are used with non-human organisms, such as in agriculture or research. For example, the organism may be a tobacco plant (e.g., Nicotiana tabacum), Caenorhabditis elegans, Drosophila, a zebrafish (Danio rerio), a pig (Sus scrofa), a Xenopus frog, or a mouse (e.g., Mus musculus) in embodiments used in research applications. In agricultural embodiments, the organism is a crop plant such as corn, wheat, maize, rapeseed, soybean, sunflower, barley, sorghum, potato, or rice. In certain agricultural embodiments, the organism is a livestock animal (e.g., cattle, horse, goat, sheep, swine, and poultry). Methods and compositions may be used to monitor treatment effectiveness and disease progression. For example, if a subject is diagnosed with cancer, the invention may be used to monitor the disease or determine effectiveness of treatment. The invention is used to detect enzymatic activity after a therapeutic treatment is administered to the subject for a prescribed time period. Effectiveness of the therapeutic treatment is determined based upon a comparison of the enzymatic activity before and after the therapeutic treatment. In an aspect of the invention, the physiological state of the subject is monitored after a course of treatment has been prescribed.

The sample may be any suitable sample from a subject. For example, the sample may be a urine sample, a breath sample, a sweat sample, or a tissue sample. Furthermore, the sample may be collected by any suitable method. For example, the sample may be excreted from the body, the sample may be obtained by liquid or tissue biopsy, and the sample may be imaged using imaging methods.

In an embodiment, the invention may be used to predict that the subject is in a pre-disease state. For example, enzymes associated with cancer may be detected, but the subject may not have any tumor growth. As such, the subject is in a pre-cancer state.

In an embodiment, an onset of the physiological state of the subject is diagnosed based on the liberated reporters detected. The physiological state may be any detectable physiological state. In certain aspects, the physiological state is a disease. In some embodiments, the disease is characterized by inflammation or atrophy. In some cases, the disease is cancer, arthritis, a trauma injury, or a muscle injury. In an example, the disease is cancer and extracellular matrix remodeling comprises development of a tumor. In another example, the disease is arthritis and extracellular matrix remodeling comprises loss of cartilage.

In an embodiment, the reporters or analytes of the present invention correspond to the portion of the substrate that has been cleaved by enzymatic activity. The reporters detected are indicative of enzymatic activity in the extracellular matrix. In particular, if the subject suffers from a disease such as cancer, composition analytes will be cleaved from the carrier at the cleavage site if proteases associated with cancer are present in the subject. In some embodiments, the detected enzymatic presence furthers indicate staging of the disease. The activity sensors may be tailored for specific disease detection and have different routes of delivery and readouts.

Any suitable delivery route may be used. For example, routes of delivery include intravenous, aerosol inhalation, subcutaneous sustained release, oral ingestion, and transdermal delivery. As a non-limiting example, aerosolized probes may retain the ability to detect protease activity and aerosol delivery of the probes may significantly accumulate in lung tumors. In a preferred embodiment, the route of delivery is intravenous delivery by an injection.

In an embodiment, the signals the sensors produce may be barcoded onto the activity sensors. Because the activity sensors are engineered, the readout may be by any suitable method. For example, the readout may be by mass spectroscopy, lateral flow, or ELISA. A bar-coded activity sensor cocktail, or composition, is assembled for each disease of interest by looking at proteases for the disease expression. The activity sensor cocktail is engineered and administered to a subject. A sample is collected from the subject, such as a urine sample, after a suitable time period, e.g. about 1 hour. The sample is then analyzed to detect the barcodes and provide results to the subject.

In certain embodiments, candidate substrates are on-target and off-target proteases. On-target proteases are proteases indicative of a certain disease or condition. Off-target proteases are indicative of a baseline enzymatic level in a subject, thereby indicating “healthy” tissue in the subject. Other off-target proteases are indicative of a disease or condition different from the target disease or condition, which may be a comorbidity or related disease producing effects similar to effects of the target disease. By including the off-target protease detection in the composition, detection is more sensitive and less likely to indicate a false positive result for the specified disease. Thus compositions may include a plurality of activity sensors, each activity sensor bearing reporters susceptible to a specific enzyme. The composition may comprise thousands of activity sensors, wherein each activity sensor is a nanosensor. Of the activity sensors in the composition, hundreds may be activity sensors directed to activity detection of a first particular enzyme, hundreds may be activity sensors directed to activity detection of a second particular enzyme, hundreds may be activity sensors directed to activity detection of a third particular enzyme, etc. As such, the composition is engineered for detection of a specific condition. The composition may include activity reporters specific to enzymes that are not dysregulated under the disease condition, detection of which may be used to establish a baseline enzyme activity level of the subject. Compositions may include an activity reporter specific to an enzyme associated with a known comorbidity to a disease or condition. For example, comorbidities to NASH are obesity and Type 2 Diabetes. As such, a composition directed to detection of NASH should include activity sensors with a plurality of reporters susceptible to cleavage by enzymes associated with obesity and a plurality of reporters susceptible to cleavage by enzymes associated with Type 2 Diabetes.

By providing specific enzymes to indicate a baseline enzyme activity level for the subject, probe for a specific disease or condition, and probe for comorbidities of that specific disease or condition, the present invention provides a therapeutic profile for the subject. The profile of the subject may be beneficial in monitoring health and determining therapeutic treatment options, should the subject be diagnosed with a disease or condition.

In an embodiment, the activity sensor comprises a carrier and a plurality of reporters. The carrier comprises one or a plurality of molecular subunits. Each of the plurality of reporters is linked to the carrier by a cleavable linker containing a cleavage site of the one or more extracellular matrix remodeling enzymes. One or more reporters are liberated upon cleavage by the one or more extracellular matrix remodeling enzymes, thereby reporting enzymatic activity.

The activity sensors are molecular machines that monitor enzyme activity. The activity sensors release reporters in the presence of specified enzymes. The reporters are detectable in a sample collected from the subject, which may be any suitable type of sample collection. For example, the sample collection may be a urine sample.

The activity sensors may have an inert core or carrier, such as a polymer core, an engineered substrate, and barcoded D-amino acid reporters or analytes. The reporters or analytes are released from the core of the activity sensors when acted on by an enzyme or protease. The released, or liberated, reporters or analytes may then be detected in a sample collected from the subject, e.g. in a urine sample.

Although enzymatic cleavage is discussed herein, other methods may be used to cleave the reporters. Cleavage by light and chemical cleavage are non-limiting examples of cleavage that do not occur from enzymatic activity. The reporter may be any suitable reporter. In certain embodiments, the reporter may be barcoded for detection purposes. In preferred embodiments, the reporter is engineered to be a substrate cleavable by a protease associated with extracellular matrix remodeling.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of detecting changes in extracellular matrix, transmembrane proteins, and/or the tissue microenvironment, the method comprising:
administering to a subject a plurality of activity sensors that release detectable reporters under conditions indicative of remodeling of cellular architecture;
detecting the detectable reporters in a sample collected from the subject; and
correlating the detectable reporters to a specific condition in the subject.

2. The method of claim 1, wherein said remodeling comprises changes in an extracellular matrix, transmembrane protein or the tissue microenvironment.

3. The method of claim 1, further comprising quantifying the detectable reporters in the sample.

4. The method of claim 1, further comprising preparing a report indicative of health status of the subject.

5. The method of claim 1, wherein the condition is cancer, fibrosis, thrombosis, arthritis, a trauma injury, or a muscle injury.

6. The method of claim 5, wherein the condition is cancer and remodeling of the extracellular matrix or tissue microenvironment comprises tumorigenesis.

7. The method of claim 5, wherein the condition is arthritis and extracellular matrix remodeling or tissue microenvironment changes comprise loss of cartilage.

8. The method of claim 5, wherein the condition is characterized by inflammation or atrophy.

9. The method of claim 1, wherein the condition is a proliferative disorder.

10. The method of claim 1, wherein the detectable reporters are detected by imaging or an immunoassay.

11. The method of claim 1, further comprising monitoring changes in disease status in the subject at a single or multiple time points, after a treatment has been administered.

12. The method of claim 1, wherein each activity sensor comprises:
a carrier comprising one or a plurality of molecular subunits; and
the plurality of reporters, each linked to the carrier by a cleavable linker containing a cleavage site of the one or more enzymes involved in remodeling of the extracellular matrix or tissue microenvironment.

13. The method of claim 12, wherein the carrier comprises a 20 to 60 kDa scaffold of linked polyethylene glycol (PEG) subunits.

14. The method of claim 13, wherein each reporter and cleavable linker comprises a polypeptide susceptible to cleavage by a protease known to be associated with a specific disease or condition associated with remodeling of the extracellular matrix or tissue microenvironment.

15. The method of claim 1, further comprising using imaging or pathology to examine a site of the remodeling.
